(19) United States
(12) Patent Application Publication
(10) Pub. No.: US 2011/0159128 A1
(43) Pub. Date: Jun. 30, 2011

(54) ALCOHOL METABOLISM ENHANCER AND ALCOHOLIC BEVERAGE

(75) Inventors: Kinya TAKAGAKI, Fukuoka (JP); Takeshi MITSUI, Chiba (JP)

(73) Assignee: TOYO SHINYAKU CO., LTD., Fukuoka-shi (JP)

(21) Appl. No.: 13/041,552

(22) Filed: Mar. 7, 2011

Related U.S. Application Data
(63) Continuation of application No. 11/721,710, filed on Sep. 25, 2009, now abandoned.

(51) Int. Cl. A61K 36/15; A61P 3/00

(52) U.S. Cl. 424/770

(57) ABSTRACT

An alcohol metabolism enhancer is made from safe plant extracts and an alcoholic beverage and causes a reduced sick feeling and hangover from drinking alcohol without causing hemolysis using safe materials derived from plants. The alcohol metabolism enhancer and the alcoholic beverage include proanthocyanidins, such as pine bark extracts.

### BACKGROUND OF THE INVENTION

1. Field of the Invention

The present invention relates to alcohol metabolism enhancers and alcoholic beverages, and in particular, to an alcohol metabolism enhancer and an alcoholic beverage that include proanthocyanidins.

2. Description of the Related Art

When ingested into the body, ethanol is oxidized by alcohol dehydrogenase (ADH) in the liver into acetaldehyde, which is further converted to acetic acid by aldehyde dehydrogenase (ALDH) and is excreted from the body.

When acetaldehyde, an alcohol metabolite, is not fully metabolized and accumulates in the body, it causes flushing, a sick feeling and other symptoms characteristic of the state of being “drunk,” which further leads to headache, nausea and other hangover symptoms.

Several plant-derived food products have been proposed that are intended to enhance alcohol metabolism and are safe for use in the body (See, for example, Japanese Patent Laid-Open Publication No. Hei 7-285881 (Patent Document 1) and Japanese Patent Laid-Open Publication No. 2002-161045 (Patent Document 2)).

Healthy alcoholic beverages have also been proposed that alleviate the harmful effects of alcohol consumption, including a sick feeling and a hangover from drinking, and detrimental effects of alcohol on heart function (See, for example, Japanese Patent Laid-Open Publication No. Sho 61-166390 (Patent Document 3)).

Although some alcohol metabolism enhancers are reported to be derived from certain types of food products traditionally known to improve the alcohol metabolism, their effects are insignificant and few are in practical use.

Meanwhile, there has been a great need for novel plant-derived alcohol metabolism enhancers that can alleviate the sick feeling and hangover from drinking by enhancing alcohol metabolism in the body and that are safe for use.

Studies have shown that numerous plant extracts have various effects on the body.

Saponins, a group of compounds traditionally used to alleviate harmful effects of alcohol consumption, including a sick feeling and a hangover as well as decreased heart function, are known to cause hemolysis even at low concentrations.

Thus, alcoholic beverages that can alleviate the sick feeling and hangover from drinking by making use of safe plant-derived materials that do not cause hemolysis are needed.

### SUMMARY OF THE INVENTION

In view of the current state of the art, the present inventors have devoted much effort to finding plant extracts that enhance alcohol metabolism and have found that proanthocyanidins, in particular pine bark extracts, serve as effective alcohol metabolism enhancers. It is this discovery that ultimately led to the present invention.

Accordingly, a preferred embodiment of the present invention is an alcohol metabolism enhancer including a proanthocyanidin.

One preferred embodiment of the present invention is the alcohol metabolism enhancer including ascorbic acid and a derivative thereof.

Another preferred embodiment is the alcohol metabolism enhancer including sesamin and an analogue thereof. The alcohol metabolism enhancer may include sesamin and an analogue thereof and also include ascorbic acid and a derivative thereof.

Another preferred embodiment is the alcohol metabolism enhancer wherein the proanthocyanidin is a pine bark extract.

The present inventors have also devoted significant effort to finding plant extracts that enhance alcohol metabolism and have found that proanthocyanidins, specifically, pine bark extracts including proanthocyanidins, function as effective alcohol metabolism enhancers.

Accordingly, another preferred embodiment of the present invention is an alcoholic beverage to which a proanthocyanidin is added.

A preferred embodiment is the healthy alcoholic beverage to which ascorbic acid and a derivative thereof are added.

Another preferred embodiment is the alcoholic beverage that includes a pine bark extract including the above-mentioned proanthocyanidin.

Another preferred embodiment is the alcoholic beverage that includes a proanthocyanidin, and effectively enhances alcohol metabolism.

Natural materials, such as pine bark extracts, function as highly safe and effective alcohol metabolism enhancers that can alleviate the sick feeling and hangover from drinking. The present invention provides such an alcohol metabolism enhancer.

Natural materials, such as pine bark extracts, function as highly safe and effective alcohol metabolism enhancers so that the present invention provides alcoholic beverages that cause a reduced sick feeling and hangover from drinking.

These and other features, elements, characteristics and advantages of the present invention will become further apparent to those skilled in the art upon reading the following description of the best mode for carrying out the invention.

### DETAILED DESCRIPTION OF PREFERRED EMBODIMENTS

Proanthocyanidins for use in the alcohol metabolism enhancer or the alcoholic beverage of the present invention are a class of compounds that are polycondensates of flavan-3-ol and/or flavan-3,4-diol structural units with a degree of polymerization of about 2 or more.

The proanthocyanidin for use in preferred embodiments of the present invention preferably includes a significant proportion of a polycondensate with a low degree of polymerization.

The polycondensate with a low degree of polymerization preferably has a degree of polymerization of about 2 to about 30 (dimer to 30-mer), more preferably a degree of polymerization of about 2 to about 10 (dimer to decamer), and still more preferably a degree of polymerization of about 2 to about 4 (dimer to tetramer). In this specification, the polycondensate with a low degree of polymerization of about 2 to about 4 is referred to as an oligomeric proanthocyanidin (OPC).

A type of polyphenols, proanthocyanidins are strong antioxidants produced by plants and are predominantly found in leaves, barks, peels and seeds. Specifically, proanthocyanidins, in particular OPCs, are found in the bark of pine, oak and Myrica rubra trees, fruit or seeds of grapes, blueberries, strawberries, avocados, false acacia and cowberries, barley, wheat, soybeans, black soybeans, cacao beans, adzuki beans, shells of horse chestnuts, husks of peanuts and ginkgo leaves. OPCs are also known to be present, though in very small amounts, in Kola nuts native to West Africa, the roots of Peruvian Rathania plant and Japanese green teas.

Human bodies cannot synthesize OPCs.

The proanthocyanidin for use in the alcohol metabolism enhancers and alcoholic beverages of preferred embodiments of the present invention may be a food material such as an extract of the above-described tree barks, fruit or seeds. Pine bark extracts are particularly preferred. Pine barks are a rich source of OPCs, among other proanthocyanidins, and are thus particularly preferred as a raw material to obtain proanthocyanidins.

An exemplary process for preparing a proanthocyanidin from a pine bark extract, a rich source of OPCs, is described below.

The pine bark extract for use in the present invention is preferably obtained from plants of the order Pinales, including Pinus Maritima, larch (Larix kaempferi), Pinus thunbergii, Pinus densiflora, Japanese white pine (Pinus parviflora), Pinus koraiensis, Pinus pumila, Pinus luchuensis, Pinus densiflora f. umbraculifera, Pinus palistris, Pinus bungeana and Anneda pine tree native to Quebec, Canada. Extracts obtained from Pinus Martima are particularly preferred.

Pinus Maritima is a coastal pine species growing in the Atlantic coastal regions of southern France. The bark of Pinus Maritima is known to contain proanthocyanidins, organic acids and other physiologically active components. Proanthocyanidins, the major components, are strong antioxidants that effectively remove active oxygen.

The pine bark extracts are obtained by extracting the above-described pine barks with water or organic solvents. Warm or hot water is used for this purpose. The organic solvent for extraction may be any organic solvent commonly used in food or pharmaceutical products, including methanol, ethanol, 1-propanol, 2-propanol, 1-butanol, 2-butanol, acetone, hexane, cyclohexane, propylene glycol, aqueous ethanol, aqueous propylene glycol, ethyl methyl ketone, glycerol, methyl acetate, ethyl acetate, diethyl ether, dichloromethane, edible fats and oils, 1,1,1,2-tetrafluoroethane and 1,1,2-trichloroethene. Water and the organic solvents may be used either individually or in combination. Hot water, aqueous ethanol and aqueous propylene glycol are particularly preferred.

While proanthocyanidins may be extracted from the pine bark by any suitable technique, they are preferably extracted by hot extraction or supercritical fluid extraction.

Supercritical fluid extraction is a process that uses a fluid in a supercritical state in which the fluid is above the critical point (critical temperature and critical pressure) at which the boundary between gas and liquid disappears. The supercritical fluid may be carbon dioxide, ethylene, propane and nitrous oxide. Of these, carbon dioxide is preferred.

Supercritical fluid extraction involves two steps: an extraction step in which desired components are extracted with a supercritical fluid, and a separation step in which the desired components are separated from the supercritical fluid. The separation step may be performed by extraction based on pressure change or temperature change or extraction using an adsorbent.

Alternatively, the supercritical fluid extraction may involve the use of entrainers. In this technique, ethanol, propanol, n-hexane, acetone, toluene, other aliphatic lower alcohols, aliphatic hydrocarbons, aromatic hydrocarbons or ketones are added to a supercritical fluid in an amount of about 2 to about 20 w/v %, and the resulting extraction fluid is used to perform supercritical fluid extraction. This technique can significantly increase the solubility of desired components, such as OPCs and catechins (which will be described later), in the extraction solvent and improve the selectivity of separation, thus enabling effective production of pine bark extracts.

Supercritical fluid extraction has the following advantages:
It can be performed at relatively low temperatures and can thus be applied to materials prone to denaturation/decomposition at high temperatures;
It does not lead to residual extraction fluid; and
It enables recycling of the solvents, eliminating the need to remove solvents and thus making the process simple.

The extraction of proanthocyanidins from the pine bark may be performed by other techniques such as liquid carbon dioxide batch process, liquid carbon dioxide reflux and supercritical carbon dioxide reflux.

Multiple extraction techniques may be combined to extract proanthocyanidins from the pine bark. The combination of different extraction techniques enables the separation of pine bark extracts with different compositions.

The pine bark extract for use in the alcohol metabolism enhancer and alcoholic beverage of preferred embodiments of the present invention can be produced by the following exemplary process, which is intended to be illustrative only and does not limit the scope of the invention in any way.

About 1 kg of the bark obtained from Pinus Maritima is immersed in about 3 L of a saturated aqueous sodium chloride solution. The solution is maintained at about 100° C. for about 30 min for extraction (Extraction step). The extract is filtered and the resulting insoluble material is washed with about 500 mL of a saturated aqueous sodium chloride solution to obtain a wash (Washing step). The extract and the wash are combined to provide a crude extract of the pine bark.

To the crude extract, about 250 mL ethyl acetate is added and then separated, and the ethyl acetate layer is collected. This is repeated 5 times. The collected ethyl acetate solutions are combined and directly added to about 200 g anhydrous sodium sulfate for dehydration. The ethyl acetate solution is then filtered and the filtrate is concentrated to one-fifth the original volume under reduced pressure. The concentrated ethyl acetate solution is poured into about 2 L chloroform and the mixture is stirred to cause precipitation.

The resulting precipitate is collected by filtration. The collected precipitate is then dissolved in about 100 mL ethyl acetate and the solution is again added to about 1 L chloroform for precipitation. This is repeated twice to perform the washing step. The entire process gives, for instance, approximately 5 g of a pine bark extract containing about 20 wt % or more OPCs (dimer to tetramer) and about 5 wt % or more catechins.

To ensure safety of the food products that use the pine bark extract of preferred embodiments of the present invention, it is preferred that the proanthocyanidins are extracted from the pine bark with ethanol or water, preferably warmed, and are concentrated using an adsorbent resin (e.g., DIAION HP-20, Sephadex-LH20 and chitin) or a ultrafiltration membrane.

Although proanthocyanidins may be present in the plant extract in any amount with no specific upper limit, their amount in the extract is typically about 90 wt % or less, preferably about 85 wt % or less, and more preferably about 80 wt % or less. The activity of proanthocyanidins is decreased when the compounds are present at too high a concentration.

The plant extracts obtained from plant materials, such as pine barks, preferably contain OPCs in an amount of at least about 20 wt %, and more preferably in an amount of at least about 30 wt % (by dry weight). In this respect, the pine bark extracts are preferred materials because of their high OPCs content.

Although the plant extracts obtained with water or ethanol may include proanthocyanidins of pentamer or higher oligomers, most of the proanthocyanidins present are smaller than the range of decamer to 20-mer because of the limited solubility of proanthocyanidins in polar solvents.

The plant extracts preferably contain at least about 5 wt % of catechins along with proanthocyanidins, in particular OPCs. The term “catechins” is the collective term for polyhydroxyflavan-3-ols. Examples of catechins include (+)-catechin, (−)-epicatechin, (+)-gallocatechin, (−)-epigallocatechin, epigallocatechin gallate and epicatechin gallate. In addition to (+)-catechin, the compound commonly referred to as “catechin” in a narrow sense, gallocatechin, afzelechin, and 3-galloyl derivatives of (+)-catechin or gallocatechin have been isolated from the extracts of pine barks and other plant materials. Catechins are known to act as an anticarcinogenic, anti-arteriosclerotic, anti-lipidotic, antihypertensive, antithrombotic, antiallergic, antiviral, antimicrobial, anticaries, antihalitosis or antioxidative agent, or have an ability to recover enteric flora, or eliminate active oxygen or free radicals. Catechins are also known to suppress the increase in the blood glucose level and act as an antidiabetic agent. Catechins increase their solubility in water in the presence of OPCs and activate OPCs. Ingesting catechins with OPCs therefore enhances the activity of OPCs.

Catechins, which increase the solubility and activity of OPCs, are preferably present in an amount of at least about 0.1 parts by weight for 1 part by weight of proanthocyanidins. It is more preferred that a plant extract containing at least about 20 wt % OPCs is prepared to include at least about 5 wt % catechins. For example, if the amount of catechins in a given pine bark extract turns out to be less than 5 wt %, then additional catechins may be added to the extract so that they account for at least about 5 wt % of the extract. Most preferably, the pine bark extract includes at least about 5 wt % catechins and at least about 20 wt % OPCs.

Proanthocyanidins, in particular OPCs, are antioxidants that not only reduce the risk of cancer, heart disease, cerebral thrombosis and other adult diseases, but also ameliorate arthritis, atopic dermatitis, pollenosis and other allergies.

In addition to having antioxidative activity, OPCs are known to act to suppress the growth of oral bacteria and decrease plaque formation, recover the elasticity of blood vessels, improve the skin texture, increase collagen production, ameliorate hyperlipidemia, prevent oxidation of blood lipoproteins by active oxygen (which otherwise causes the oxidized fat to build up in the inner wall of vessels, forming cholesterol deposits), replace Vitamin E decomposed by the action of active oxygen, and increase the activity of Vitamin E.

The alcohol metabolism enhancer of the present invention includes proanthocyanidins preferably in an amount of about 0.00001 wt % to about 50 wt %, more preferably in an amount of about 0.001 wt % to about 40 wt %, still more preferably in an amount of about 0.01 wt % to about 20 wt %, and most preferably in an amount of about 0.01 wt % to about 5 wt % (by dry weight) in the composition. It is preferably administered to an adult (weighing 60 kg) at a daily dose of about 10 mg to about 2000 mg, more preferably at a daily dose of about 20 mg to about 1000 mg, and most preferably at a daily dose of about 20 mg to about 300 mg. The alcohol metabolism enhancer administered at a daily dose of about 10 mg/adult or less cannot provide the desired effects, whereas increasing the dose to about 2000 mg/adult or more does not correspondingly increase the intended effects.

The alcohol metabolism enhancer of preferred embodiments of the present invention effectively promotes alcohol metabolism and prevents the sick feeling and hangover from drinking whether it is ingested before or after the alcohol consumption. Even if ingested as long as 90 minutes prior to alcohol consumption, the alcohol metabolism enhancer still promotes alcohol metabolism and effectively prevents the sick feeling and hangover from drinking.

The alcoholic beverage of a preferred embodiment of the present invention includes proanthocyanidins preferably in an amount of about 0.00001 wt % to about 50 wt %, more preferably in an amount of about 0.001 wt % to about 40 wt %, still more preferably in an amount of about 0.01 wt % to about 20 wt %, and most preferably in an amount of about 0.01 wt % to about 5 wt % (by dry weight) in the composition. It is preferably ingested by an adult (weighing about 60 kg) so that the daily intake of proanthocyanidin is from about 10 mg to about 2000 mg, more preferably from about 20 mg to about 1000 mg, and most preferably from about 20 mg to about 300 mg. The alcoholic beverage including too little proanthocyanidin cannot provide the desired effects, whereas the same beverage including about 40 wt % or more proanthocyanidin is susceptible to color change and readily forms precipitation.

Aside from proanthocyanidins, the alcohol metabolism enhancer and the alcoholic beverage of the present invention may include other components commonly used in quasi drugs. These components are added in amounts that do not affect the advantages of each composition.

Examples of such components include water, other active ingredients, other oil materials, humectants, surfactants, UV absorbers, absorption enhancers, flavors, pigments, preservatives, thickeners, chelators and antiseptic/mildew-proofing agent.

Examples of other active ingredients include active oxygen scavengers, antioxidants, anti-inflammatory agent, antihistamines, antipruritics, bactericides, vitamins and hormones.

Antioxidants may be added to increase the stability of proanthocyanidins. The antioxidants prevent oxidation of proteins and lipids in the body and improve and protect skin texture.

Examples of the antioxidants include Vitamin A and other carotenoids, Vitamin B, ascorbic acid, Vitamin E, derivatives and salts thereof, L-cysteine and derivatives and salts thereof, riboflavin, SOD, mannitol, tryptophan, histidine, quercetin, gallic acid and derivatives thereof, tea leave extracts, sesamin and analogues thereof, and extracts such as glutathione yeast extracts.

Of these, ascorbic acid and derivatives and salts thereof increase the stability of proanthocyanidins. They function not only to increase the activity of proanthocyanidins, but also to facilitate decomposition of alcohol and aldehyde in liver.

Examples of the derivatives of ascorbic acid include ascorbyl-2-phosphate and ascorbyl-2-glucoside. Examples of the salts include sodium salts and potassium salts.

Ascorbic acid may be added to the alcohol metabolism enhancer and the alcoholic beverage of a preferred embodiment of the present invention such that the ratio by weight of ascorbic acid to proanthocyanidins is preferably in the range of about 1:0.1 to about 1:500, and more preferably in the range of about 1:0.2 to about 1:200.

Sesamin, a lignan derivative found in sesame seed extracts, and its analogues are also effective antioxidants and can stabilize proanthocyanidins. Sesamin also functions to improve liver functions and facilitate decomposition of alcohol and aldehyde. The compound thus synergistically improves alcohol metabolism.

Examples of sesamin analogues include sesamolin, sesaminol, sesamol, episesamin, pyrdinol, and sesangolin. Sesame seed extracts (sesame lignan) containing these analogues may also be used.

Sesamin and sesamin analogues are added so that the ratio by weight of sesamin to proanthocyanidins is preferably in the range of about 1:0.01 to about 1:50, and more preferably in the range of about 1:0.02 to about 1:20.

Sesamin and analogues thereof may be used in combination with ascorbic acid and derivatives and salts thereof to achieve a synergistic effect.

When necessary, a food composition including the alcohol metabolism enhancer may be mixed with excipients, bulking agents, binders, thickeners, emulsifiers, colors, flavors, food additives, flavoring materials and other additives.

Examples of the food additives include royal jelly, vitamins, proteins, egg shell calcium and other calcium sources, chitosan, lecithin, chlorella powder, Angelica keiskei plant powder, mulukhiyya powder and other nutrients. Examples of the flavoring materials include stevia powder, green tea leave powder, lemon powder, honey, reduced malt sugar, lactose and glucose solution.

These ingredients may be formulated in the form of capsules, such as hard capsules and soft capsules, tablets, pills, powders, granules, tea-like preparations, teabag-like pouches, candies, liquids or pastes. These preparations may be directly eaten or drunk, or they may be dissolved in water or hot water for drinking.

The alcoholic beverage of preferred embodiments of the present invention may be obtained by adding a plant extract including proanthocyanidins directly to beer, whisky, sake, shochu, gin, rum, vodka and other alcoholic beverages, or by adding the extract during the fermentation or distillation process. Since the proanthocyanidins are susceptible to oxidation or decomposition during fermentation or distillation processes, plant extracts are preferably added to the alcoholic beverages after fermentation or distillation.

The present invention will now be described in detail with reference to Examples.

### Example 1

18 male SD rats, aged 10 weeks (Kyudo Co., Ltd.), were fed a solid diet (Oriental Yeast Co., Ltd.) for 1 week and were subsequently fasted for 12 hours.

Rats were each tube-fed 5 mL of a 0.1 wt % aqueous solution of a pine bark extract containing 40 wt % proanthocyanidins (20 wt % OPC content, FLAVANGENOL, Toyo Shinyaku Co., Ltd.). 30 minutes after the administration of the pine bark extract, a 15 wt % aqueous ethanol was orally administered so that the total ethanol amounted to 1.0 g/kg. Blood samples were collected 0.5, 1 and 6 hours after the administration of ethanol and were analyzed for blood ethanol and acetaldehyde levels using a commercially available analysis kit (F-kit ethanol, F-kit acetaldehyde, Beringer-Mannheim Co., Ltd.).

The results are shown in Table 1.

### Example 2

The experiment was conducted in the same manner as in Example 1, except that 0.1 wt % ascorbic acid was added to the aqueous solution of the pine bark extract. The blood ethanol and acetaldehyde levels were measured.

The results are shown in Table 1.

### Comparative Example 1

The experiment was conducted in the same manner as in Example 1, except that the pine bark extract was not added. The blood ethanol and acetaldehyde levels were measured. The results are shown in Table 1.

### Comparative Example 2

The experiment was conducted in the same manner as in Example 1, except that the pine bark extract was replaced by a grape seed extract containing 38 wt % proanthocyanidins (Kikkoman Co., Ltd.). The blood ethanol and acetaldehyde levels were measured.

The results are shown in Table 1.

TABLE 1

| | Ethanol Conc.(mg/L) | | | | Acetaldehyde Conc.(mg/L) | | | |
|---|---|---|---|---|---|---|---|---|
| | | | Comparative | Comparative | | | Comparative | Comparative |
| Time (hr) | Example 1 | Example 2 | Example 1 | Example 2 | Example 1 | Example 2 | Example 1 | Example 2 |
| 0 | 0 | 0 | 0 | 0 | 0 | 0 | 0 | 0 |
| 0.5 | 751 ± 70.6 | 731 ± 68.2 | 780 ± 69.5 | 772 ± 77.1 | 30.1 ± 2.9 | 28.1 ± 1.6 | 32.1 ± 2.6 | 31.1 ± 2.1 |
| 1.0 | 591 ± 40.2 | 541 ± 46.7 | 751 ± 44.2 | 684 ± 31.2 | 22.4 ± 1.1 | 20.4 ± 1.5 | 25.1 ± 1.5 | 23.7 ± 2.0 |
| 6.0 | 34 ± 6.5 | 14 ± 4.2 | 110 ± 46.8 | 51 ± 10.1 | 5.5 ± 1.5 | 3.1 ± 1.2 | 10.4 ± 5.1 | 7.1 ± 1.0 |

Mean ± SE

As can be seen from the results of Table 1, the blood concentrations of ethanol and acetaldehyde decreased faster in each of the groups administered proanthocyanidins with alcohol than in the group given no proanthocyanidins, indicating faster alcohol metabolism in the proanthocyanidin groups. The ethanol and acetaldehyde concentrations decreased faster in the groups given the pine bark extract than in the group given the grape seed extract.

The combination of proanthocyanidins and ascorbic acid proved to be most effective.

### Example 3

5 healthy male subjects were asked to refrain from drinking alcohol for 1 week.

Each subject ingested 3 tablets of Food Product 1 including a pine bark extract with 40 wt % proanthocyanidin content as shown in Table 2. Starting at the beginning of the test (0 min), the subjects drank whisky-and-water containing 6 wt % alcohol over a time period of 2 hours so that the total alcohol consumption amounted to 2 g alcohol/1 kg body weight.

The subjects were asked to evaluate their health conditions 3 hours after they had finished alcohol consumption and on the following morning.

Each tablet of the food product weighed 100 mg (in all examples).

The results are shown in Table 3.

### Example 4

The same subjects consumed alcohol in the same manner as in Example 3, except that following one-week abstinence period, each ingested 3 tablets of Food Product 2, which contained a pine bark extract with 40 wt % proanthocyanidin content and ascorbic acid (Table 2). The subjects were asked to evaluate their health conditions 3 hours after the end of the alcohol consumption period and on the following morning.

The results are shown in Table 3.

### Example 5

The same subjects consumed alcohol in the same manner as in Example 3, except that following one-week abstinence period, each ingested 3 tablets of Food Product 3, which contained a pine bark extract with 40 wt % proanthocyanidin content and a sesame seed extract as shown in Table 2. The subjects were asked to evaluate their health conditions 3 hours after the end of the alcohol consumption period and on the following morning.

The results are shown in Table 3.

### Comparative Example 3

The same subjects consumed alcohol in the same manner as in Example 3, except that following one-week abstinence period, each ingested 3 tablets of Food Product 4 as shown in Table 2. The subjects were asked to evaluate their health conditions 3 hours after the end of the alcohol consumption period and on the following morning.

The results are shown in Table 3.

TABLE 2

| | | Food | Food | Food | Food |
|---|---|---|---|---|---|
| | | Product | Product | Product | Product |
| Ingredients | | 1 | 2 | 3 | 4 |
| Pine bark extract | | 20 | 20 | 20 | — |
| Ascorbic acid | | — | 20 | — | — |
| Sesame seed extract * 1 | | — | — | 20 | — |
| Additives | Crystalline | 20 | 10 | 10 | 40 |
| | cellulose | | | | |
| | Sucrose ester | 5 | 5 | 5 | 5 |
| | Silicon dioxide | 2 | 2 | 2 | 2 |
| | Egg shell calcium | 53 | 43 | 43 | 53 |

Units are parts by weight.
* 1: contains 95% sesamin and its analogues.

TABLE 3

| | | | | | Comparative |
|---|---|---|---|---|---|
| | | Example | Example | Example | Example |
| | | 3 | 4 | 5 | 3 |
| 3 hours later | Headache | 2 | 2 | 1 | 4 |
| | Nausea | 1 | 0 | 1 | 3 |
| | Sleepy | 1 | 0 | 0 | 2 |
| | Chill | 0 | 0 | 0 | 2 |
| Following | Headache | 1 | 0 | 1 | 4 |
| morning | Nausea | 0 | 0 | 0 | 2 |
| | Sluggishness | 2 | 2 | 0 | 4 |

As can be seen from the results of Table 3, symptoms of sick feeling and hangover from drinking alcohol were significantly reduced in each of the groups that ingested the pine extract prior to alcohol consumption, as compared to the control group.

Addition of ascorbic acid, sesamin and analogues thereof significantly improved the result.

### Example 6

5 healthy male subjects were asked to refrain from drinking alcohol for 1 week.

Starting at the beginning of the test (0 min), each subject drank whisky-and-water containing 6 wt % alcohol over a time period of 2 hours so that the total alcohol consumption amounted to 2 g alcohol/1 kg body weight.

After the alcohol consumption period, the subjects ingested 3 tablets of Food Product 1 containing a pine bark extract with 40 wt % proanthocyanidin content as shown in Table 2.

The subjects were asked to evaluate their health conditions on the following morning.

The results are shown in Table 4.

### Example 7

Following one-week abstinence period, the same subjects consumed alcohol in the same manner as in Example 6.

After the alcohol consumption period, the subjects ingested 3 tablets of Food Product 2, which contained a pine bark extract with 40 wt % proanthocyanidin content and ascorbic acid as shown in Table 2.

The subjects were asked to evaluate their health conditions on the following morning.

The results are shown in Table 4.

### Example 8

Following one-week abstinence period, the same subjects consumed alcohol in the same manner as in Example 6.

After the alcohol consumption period, the subjects ingested 3 tablets of Food Product 3, which contained a pine bark extract with 40 wt % proanthocyanidin content and a sesame seed extract as shown in Table 2.

The subjects were asked to evaluate their health conditions on the following morning.

The results are shown in Table 4.

### Comparative Example 4

Following one-week abstinence period, the same subjects consumed alcohol in the same manner as in Example 6.

After the alcohol consumption period, the subjects ingested 3 tablets of Food Product 4 as shown in Table 2.

The subjects were asked to evaluate their health conditions on the following morning.

The results are shown in Table 4.

TABLE 4

| | | | | | Comparative |
|---|---|---|---|---|---|
| | | Example | Example | Example | Example |
| | | 6 | 7 | 8 | 4 |
| Following | Headache | 2 | 1 | 1 | 4 |
| morning | Nausea | 2 | 1 | 0 | 3 |
| | Sluggishness | 2 | 1 | 1 | 4 |

As can be seen from the results of Table 4, symptoms of sick feeling and hangover from drinking alcohol were significantly reduced in each of the groups that ingested the pine extract after alcohol consumption, as compared to the control group.

Addition of ascorbic acid, sesamin and analogues thereof (present in the sesame seed extract) significantly improved the result.

### Example 9

### Evaluation of Alcohol Metabolism by Breath Alcohol Concentration

The effects of ingesting a shochu liquor containing 25% alcohol and tablets containing 30 mg/tablet of a pine bark extract on alcohol metabolism were assessed in the manner described below. The tablet weighed 250 mg each and contained 30 mg of a pine bark extract (the same extract used in Example 1), crystalline cellulose, sucrose ester, reduced malt sugar and trehalose as excipients.

Using an alcohol detector (Central Automotive Products Co., Ltd.), the breath alcohol concentration was measured in 5 subjects prior to alcohol consumption. Upon confirmation that the measurement was 0, each subject drank 60 mL shochu liquor within 5 minutes. The breath alcohol concentration of each subject was measured 5 times after 30, 60, 120, and 150 minutes of Shochu consumption. It was measured for 5 times at each instance.

At about the same time on the following day, the same experiment was conducted on the same subjects, except that each subject ingested 4 tablets containing the pine bark extract 90 minutes before shochu consumption. The breath alcohol concentration was measured in the same manner as the day before.

The subjects ingested 4 tablets of the pine bark extract 60 minutes before shochu consumption on Day 3 and 30 minutes before shochu consumption on Day 4. The breath alcohol concentration was measured and the average was taken in the same manner.

The results are shown in Table 5.

TABLE 5

| | Before | | | | |
|---|---|---|---|---|---|
| Timing of tablet | shochu | Time after shochu consumption (min) | | | |
| ingestion | consumption | 30 | 60 | 120 | 150 |
| — | 0 | 0.16 | 0.14 | 0.07 | 0.01 |
| 90 min before sho- | | | | | |
| chu consumption | 0 | 0.19 | 0.09 | 0.04 | 0.00 |
| 60 min before sho- | | | | | |
| chu consumption | 0 | 0.20 | 0.09 | 0.02 | 0.00 |
| 30 min before sho- | | | | | |
| chu consumption | 0 | 0.25 | 0.11 | 0.02 | 0.00 |

Units are mg/L

As can be seen from the results of Table 5, the breath alcohol concentration decreased faster when the subjects ingested the proanthocyanidin-containing pine bark extract of the present invention than when they did not. The results indicate that ingesting the proanthocyanidin-containing pine bark extract of the present invention before alcohol consumption facilitates alcohol metabolism and helps prevent sick feeling and hangover from drinking.

### Example 10

18 male SD rats, aged 10 weeks (Kyudo Co., Ltd.), were fed a solid diet (Oriental Yeast Co., Ltd.) for 1 week and were subsequently fasted for 12 hours.

A pine bark extract containing 40 wt % proanthocyanidins (20 wt % OPC content, FLAVANGENOL, Toyo Shinyaku Co., Ltd.) was added to an aqueous solution containing 15 wt % ethanol to a final concentration of 0.005 wt %. The aqueous ethanol solution was orally administered to rats so that the total ethanol amounted to 1.0 g/kg. Blood samples were collected 0.5, 1 and 6 hours after the administration of ethanol and were analyzed for blood ethanol and acetaldehyde levels using a commercially available analysis kit (F-kit ethanol, F-kit acetaldehyde, Beringer-Mannheim Co., Ltd.).

The results are shown in Table 6.

### Example 11

The experiment was conducted in the same manner as in Example 10, except that ascorbic acid was added to the aqueous ethanol solution of the pine bark extract to a final concentration of 0.005 wt %. The blood ethanol and acetaldehyde levels were measured.

The results are shown in Table 6.

### Comparative Example 5

The experiment was conducted in the same manner as in Example 10, except that the pine bark extract was not added. The blood ethanol and acetaldehyde levels were measured.

The results are shown in Table 6.

### Comparative Example 6

The experiment was conducted in the same manner as in Example 10, except that the pine bark extract was replaced by a grape seed extract containing 38 wt % proanthocyanidins (Kikkoman Co., Ltd.). The blood ethanol and acetaldehyde levels were measured.

The results are shown in Table 6.

TABLE 6

| | Ethanol Conc. (mg/L) | | | | Acetaldehyde Conc. (mg/L) | | | |
|---|---|---|---|---|---|---|---|---|
| | | | Comparative | Comparative | | | Comparative | Comparative |
| Time (hr) | Example 10 | Example 11 | Example 5 | Example 6 | Example 10 | Example 11 | Example 5 | Example 6 |
| 0 | 0 | 0 | 0 | 0 | 0 | 0 | 0 | 0 |
| 0.5 | 711 ± 77.1 | 701 ± 66.8 | 780 ± 69.5 | 750 ± 67.1 | 28.2 ± 3.0 | 27.2 ± 4.0 | 32.1 ± 2.6 | 30.2 ± 1.3 |
| 1.0 | 551 ± 50.2 | 520 ± 40.2 | 751 ± 44.2 | 654 ± 38.8 | 20.1 ± 2.1 | 16.1 ± 2.7 | 25.1 ± 1.5 | 23.4 ± 1.8 |
| 6.0 | 20 ± 7.8 | 12 ± 6.9 | 110 ± 46.8 | 31 ± 12.1 | 3.4 ± 1.5 | 2.1 ± 1.5 | 10.4 ± 5.1 | 5.2 ± 2.1 |

Mean ± SE

As can be seen from the results of Table 6, the blood concentrations of ethanol and acetaldehyde decreased faster in each of the groups administered proanthocyanidins with alcohol than in the group given no proanthocyanidins, indicating faster alcohol metabolism in the proanthocyanidin groups. The ethanol and acetaldehyde concentrations decreased faster in the groups given the pine bark extract than in the group given the grape seed extract.

The combination of proanthocyanidins and ascorbic acid proved to be most effective.

### Example 12

5 healthy male subjects were asked to refrain from drinking alcohol for 1 week.

An alcoholic beverage was prepared by adding 0.6 g of a pine bark extract with 40 wt % proanthocyanidin content to 300 mL of whisky (34 wt % alcohol, Suntory Co., Ltd.).

Starting at the beginning of the test (0 min), the subjects drank whisky-and-water prepared from the alcoholic beverage (6 wt % alcohol content) over a time period of 2 hours so that the total alcohol consumption amounted to 3 g alcohol/1 kg body weight.

The subjects were asked to evaluate their health conditions 3 hours after they had finished alcohol consumption and on the following morning.

The results are shown in Table 7.

### Example 13

The same subjects consumed alcohol in the same manner as in Example 12, except that following one-week abstinence period, each subject was given whisky-and-water containing 0.6 g each of the pine bark extract (the same extract used in Example 12) and ascorbic acid.

The subjects were asked to evaluate their health conditions 3 hours after the end of the alcohol consumption period and on the following morning.

The results are shown in Table 7.

### Comparative Example 7

The same subjects consumed alcohol in the same manner as in Example 12, except that following one-week abstinence period, each subject was given whisky-and-water containing no pine bark extract. The subjects were asked to evaluate their health conditions 3 hours after the end of the alcohol consumption period and on the following morning.

The results are shown in Table 7.

### Comparative Example 8

The same subjects consumed alcohol in the same manner as in Example 12, except that following one-week abstinence period, each was given red wine containing no pine bark extract, rather than whisky-and-water. The subjects were asked to evaluate their health conditions 3 hours after the end of the alcohol consumption period and on the following morning.

The results are shown in Table 7.

TABLE 7

| | | Example | Example | Comparative | Comparative |
|---|---|---|---|---|---|
| | | 12 | 13 | Example 7 | Example 8 |
| 3 hours | Headache | 2 | 1 | 4 | 4 |
| later | Nausea | 2 | 1 | 3 | 1 |
| | Sleepy | 0 | 0 | 1 | 2 |
| | Chill | 0 | 0 | 2 | 1 |
| Following | Headache | 2 | 0 | 4 | 4 |
| morning | Nausea | 0 | 0 | 2 | 1 |
| | Sluggishness | 2 | 2 | 4 | 4 |

As can be seen from the results of Table 7, symptoms of sick feeling and hangover from drinking alcohol were significantly reduced in each of the groups that ingested the alcoholic beverage of the present invention containing the pine bark extract, as compared to the groups given other types of alcoholic beverage containing no pine bark extract. The combination of proanthocyanidins and ascorbic acid proved to be most effective.

The comparison with red wine, an alcoholic beverage known to contain proanthocyanidins, revealed that better results are obtained in an alcoholic beverage to which proanthocyanidins have been added than in an alcoholic beverage in which proanthocyanidins are naturally present.

Some subjects pointed out that the flavor of the alcoholic beverage of the present invention had been improved, suggesting that the beverage has improved palatability.

### Examples 14 through 16

15 healthy male subjects were asked to refrain from drinking alcohol for 1 week.

The subjects were divided into 3 groups of 5. Starting at the beginning of the test (0 min), the 3 groups drank beer (4 wt % alcohol), red wine (6 wt % alcohol) and shochu-and-water (6 wt % alcohol), each containing 0.01 wt % of a pine bark extract with 40 wt % proanthocyanidin content, respectively, over a time period of 2 hours, so that the total alcohol consumption amounted to 3 g alcohol/1 kg body weight.

The subjects were asked to evaluate their health conditions 3 hours after they had finished alcohol consumption and on the following morning.

The results are shown in Table 8.

### Comparative Examples 9 through 11

The same subjects consumed alcohol in the same manner as in Examples 14 through 16, except that following the one-week abstinence period, the 3 groups were given respective alcoholic beverages containing no pine bark extract. The subjects were asked to evaluate their health conditions 3 hours after the end of the alcohol consumption period and on the following morning.

The results are shown in Table 8.

TABLE 8

| | | | Comparative | | Comparative | | Comparative |
|---|---|---|---|---|---|---|---|
| | | Example 14 | Example 9 | Example 15 | Example 10 | Example 16 | Example 11 |
| Alcohol beverage | | Beer | | Red wine | | Shochu | |
| 3 hours later | Headache | 1 | 2 | 1 | 3 | 1 | 4 |
| | Nausea | 0 | 2 | 1 | 2 | 1 | 2 |
| | Sleepy | 0 | 0 | 0 | 1 | 0 | 1 |
| | Chill | 0 | 1 | 0 | 1 | 0 | 0 |
| Following | Headache | 0 | 2 | 0 | 4 | 0 | 2 |
| morning | Nausea | 0 | 0 | 0 | 1 | 0 | 1 |
| | Sluggishness | 1 | 2 | 2 | 3 | 1 | 4 |

As can be seen from the results of Table 8, symptoms of sick feeling and hangover from drinking alcohol were significantly reduced in each of the 3 groups given different alcoholic beverages containing the pine bark extract. This suggests increased alcohol metabolism in each group.

### Example 17

### Evaluation of Alcohol Metabolism by Breath Alcohol Concentration

An alcoholic beverage was prepared by dissolving 500 mg of a pine bark extract (the same extract used in Example 1) in 300 ml of shochu liquor (25% alcohol). The alcoholic beverage and a pine bark extract-free shochu (control beverage) were evaluated in the following manner.

Using an alcohol detector (Central Automotive Products Co., Ltd.), the breath alcohol concentration was measured in 3 subjects prior to alcohol consumption. Upon confirmation that the measurement was 0, each subject drank 60 mL of the control beverage within 5 minutes.

The breath alcohol concentration of each subject was measured 5 times after 30, 60, 120, and 150 minutes of Shochu consumption. It was measured for 5 times at each instance.

At about the same time on the following day, the same experiment was conducted on the same subjects, except that each subject was given the alcoholic beverage, rather than the control beverage. The breath alcohol concentration was measured in the same manner as the day before.

Data shown in Table 9 are averages of the 3 subjects

TABLE 9

| | Before | | | | |
|---|---|---|---|---|---|
| | alcohol | Time after alcohol consumption (min) | | | |
| | consumption | 30 | 60 | 120 | 150 |
| Control beverage | 0 | 0.18 | 0.17 | 0.07 | 0.12 |
| Alcohol beverage | 0 | 0.16 | 0.11 | 0.05 | 0 |

Units are mg/L.

As can be seen from the results of Table 9, the breath alcohol concentration decreased faster for the alcoholic beverage of the present invention, which contained the pine bark extract containing proanthocyanidins, than for the control beverage. The results indicate that the alcoholic beverage of the present invention facilitates decomposition of alcohol and helps prevent sick feeling and hangover that may otherwise result from drinking the alcoholic beverage.

The alcohol metabolism enhancer of the present invention is expected to find wide application in the field of food production.

The alcoholic beverage of the present invention is expected to find wide application in the field of brewing.

While preferred embodiments of the present invention have been described above, it is to be understood that variations and modifications will be apparent to those skilled in the art without departing the scope and spirit of the present invention. The scope of the present invention, therefore, is to be determined solely by the following claims.

What is claimed is:

1. A method for enhancing alcohol metabolism using an alcohol metabolism enhancer comprising a proanthocyanidin which is a pine bark extract.

2. The method for enhancing alcohol metabolism according to claim 1, wherein the alcohol metabolism enhancer further comprises ascorbic acid, ascorbyl-2-phosphate, or ascorbyl-2-glucoside.

3. The method for enhancing alcohol metabolism according to claim 2, wherein a ratio by weight of the ascorbic acid, the ascorbyl-2-phosphate, or the ascorbyl-2-glucoside to proanthocyanidin is in the range of about 1:0.1 to about 1:500.

4. The method for enhancing alcohol metabolism according to claim 1, wherein the alcohol metabolism enhancer further comprises sesamin, sesamolin, sesaminol, sesamol, episesamin, pyrdinol, or sesangolin.

5. The method for enhancing alcohol metabolism according to claim 4, wherein a ratio by weight of the sesamin, the sesamolin, the sesaminol, the sesamol, the episesamin, the pyrdinol, or the sesangolin to proanthocyanidin is in the range of about 1:0.02 to about 1:20.

6. The method for enhancing alcohol metabolism according to claim 1, wherein the proanthocyanidin is present in the alcohol metabolism enhancer in an amount of about 0.01 wt % to about 5.0 wt % by dry weight.

7. The method for enhancing alcohol metabolism according to claim 1, wherein the proanthocyanidin in the alcohol metabolism enhancer is ingested at a daily amount of about 20 mg to about 300 mg.
